(19) United States
(12) Patent Application Publication
(10) Pub. No.: US 2025/0161531 A1
(43) Pub. Date: May 22, 2025

(54) DEVICE AND METHOD FOR CREATING LOCALISED DEGRADATION IN BIOMATERIALS

(71) Applicant: Royal College of Surgeons in Ireland, Dublin (IE)

(72) Inventors: Zuzana Kocí, Nová Lesná (SK); Fergal O’Brien, Dublin (IE); William Lackington, St. Gallen (CH); Alan Hibbitts, Naas (IE); Alan Ryan, Galway (IE); Simon James Archibald, Pennington, NJ (US)

(21) Appl. No.: 18/948,762

(22) Filed: Nov. 15, 2024

Related U.S. Application Data
(63) Continuation of application No. PCT/EP2024/082553, filed on Nov. 15, 2024.
(60) Provisional application No. 63/601,898, filed on Nov. 22, 2023.

(51) Int. Cl. A61L 27/24; A61B 17/00; A61B 17/11; A61L 27/58

(52) U.S. Cl. CPC A61L 27/24; A61B 17/1128; A61L 27/58; A61B 2017/00004; A61B 2017/00526; A61L 2400/18; A61L 2430/32

(57) ABSTRACT

An implantable medical device, and a method of manufacturing the same, the device including a bioresorbable conduit including an exterior surface including one or more weakened areas configured to degrade preferentially to the remainder of the bioresorbable conduit, and an interior surface.

### CROSS-REFERENCE TO RELATED APPLICATIONS

This application is a continuation of and claims priority to co-pending PCT International Application No. PCT/EP2024/082553, filed Nov. 15, 2024, and claims priority to U.S. Provisional Patent Application Ser. No. 63/601,898, filed Nov. 22, 2023, the disclosures of which are incorporated herein by reference in their entireties.

### FIELD OF THE INVENTION

The present disclosure relates to an implantable medical device and method of manufacturing said device, and more specifically an implantable medical device with areas of bioresorbable material which degrade preferentially in vivo.

### BACKGROUND

Poor nutrient and gas exchange are significant limiting factors for tissue growth in many biomaterials across various tissue engineering applications. Vascularisation plays a fundamental role in supporting the function of organs and tissues through supplying of blood, oxygen, and other nutrients. For example, vascularisation improves peripheral nerve regeneration on several levels. Firstly, by increasing nutrient supply and debris removal, vascularisation favours long-term survival of highly metabolically active neuronal and glial cells. Secondly, blood vessels also serve as tracks for Schwann cells that migrate along and later guide axonal growth. Finally, endothelial cells contribute to nerve regeneration by synthetizing several neurite supportive factors and expressing vitronectin, heparin sulphate proteoglycans and other glycoproteins that are involved in neurite activity and growth.

Within the body, most cells reside within around 100-200 μm from the nearest vascular source to ensure sufficient delivery of oxygen by diffusion. Lack of vascularisation was reported as the most common reason for implant failure.

Blood vessels have long been known to precede Schwann cell migration as well as axonal extension. Studies suggest that there is extensive evidence that the architecture and functionality of capillaries facilitate axonal regeneration.

In order to increase the chances of biomaterial survival, many attempts have been made to increase vascularisation of such biomaterials, for example, by using vascularised conduits, using vasogenic growth factors, using conduits seeded with endothelial cells, using decellularized whole organs, and fabricating channels that mimic blood vessel network.

Vascularised conduits have been shown to be superior to non-vascularised conduits (for example, in nerve grafting). However, they are limited by the donor availability, or require time for pre-implantation which includes two surgeries and thus increases the risk of complications.

Although using vascular endothelial growth factor (VEGF) has proven successful and resulted in higher capillary number and blood vessels density it requires extensive optimization, evaluation of any side effects, and increased product costs.

Seeding conduits with endothelial cell co-culture is a complex task and does not always lead to formation of capillary networks. Moreover, this approach requires patient's own tissue for endothelial cell harvest and is hindered by increased price and the need for immediate implantation with no storage option.

Because organs contain complex and ordered vasculature by nature, they can be decellularized to be used as matrices for tissue regeneration. However, the optimization of donor organ type, species, age and decellularization method is not trivial and there is a risk of immune reaction and rejection.

Although these methods have their merits, there are continuing challenges. As such, there remains a need for further methods to increase vascularization of biomaterials, and subsequently improve overall implantation outcomes and implant survival.

### SUMMARY OF INVENTION

The present invention provides a rapid, robust, precise and high throughput device and method that controls the degradation of implanted biomaterials and improves tissue repair. The device and method of the present invention create areas in bioresorbable material which degrade preferentially in vivo, thus stimulating blood vessel ingrowth and improving regeneration outcomes.

According to a first aspect of the present invention there is provided an implantable medical device. The device comprising a bioresorbable conduit. The bioresorbable conduit comprising: an exterior surface; and an interior surface. The exterior surface comprises one or more weakened areas configured to degrade preferentially to the remainder of the bioresorbable conduit.

Advantageously, the first aspect of the present invention provides localised and controlled degradation of the bioresorbable conduit without compromising overall structural integrity. This allows for the creation of a localised concentration gradient, which subsequently allows for the passage of substances (e.g., ions, small molecules, proteins, fluids, etc.) through the material at the desired time and rate (e.g. gas exchange, growth factor chemotaxis). Therefore, the first aspect of the present invention enables not only control of the time and rate of degradation, but also the time and rate of vascular cell infiltration and vascularisation. The first aspect of the present invention further supports blood vessel ingrowth into the structure for improved tissue repair and enables rapid vascularisation at a greater rate than that which would occur if the degradation of the conduit main structural elements were the limiting factor, while providing a supportive environment for tissue regeneration.

By providing weakened areas which are configured to degrade preferentially to the remainder of the bioresorbable conduit, space for vascular cell infiltration is provided and nutrient exchanged is enabled, while the overall biomaterial integrity is not compromised and continues to provide physical support for regenerating tissue. The weakened areas with the first stage of preferential degradation also allow vascular infiltration to augment regeneration. This is then followed by a later, second stage of degradation of the biomaterial as new regenerated tissue is formed, which ultimately replaces all remnants of the biomaterial. The weakening of the biomaterial in specific areas achieves a controlled two stage degradation in order to facilitate targeted tissue repair. The weakening of the biomaterial in specific areas may achieve multiple stages of degradation and is not limited to only two stages of degradation. More than two stages of degradation may be achieved with different levels of weakening of different weakened areas or different densities of weakened areas. Thus, the present invention provides a solution for a major shortfall of tissue engineered biomaterials. This is in contrast to implantable medical devices which degrade in an uncontrolled manner over time.

Furthermore, in an example embodiment, opening space for vascular cell infiltration ultimately results in increased vessel growth especially in the outer shell of treated material that corresponds to the extrinsic vascularisation system of vasa nervorum in epineurium. Increased vessel growth is key for regeneration especially over longer distances. Moreover, increased vessel growth in epineurium is strongly correlated with increased amount of myelinated axons. Thus, the present invention improves the ability of nerve guidance conduits to repair larger nerve gaps and might replace the need for autografts which are limited by donor availability and morbidity.

In the implantable medical device, the one or more weakened areas of the bioresorbable conduit may comprise surface depressions. In this way, the process during which such depressions are enlarged into holes (due to preferential degradation) provides sufficient time for host cell infiltration and, for example, restructuring of an internal matrix. Furthermore, by the weakened areas merely being depressions in the exterior surface, the overall integrity of the conduit is preserved and provides a shielded environment from excess immune cells and fibroblasts.

In the implantable medical device, the one or more weakened areas may each have a diameter of 50-2000 μm.

In the implantable medical device, one or more of the weakened areas may be obtainable by chemically-treating, mechanically-treating or heat treating the exterior surface. In this way, the one or more weakened areas may be provided in a manner which creates weakened areas or depressions, but not through holes. This ensures that preferential degradation occurs while the overall biomaterial integrity is not compromised and continues to provide physical support for regenerating tissue.

In the implantable medical device, one or more of the weakened areas may be obtainable by laser-treating the exterior surface. In this way, the one or more weakened areas may be provided in rapid, robust, precise and high throughput manner. The laser used for the laser treatment may be a non-invasive, single-step, highly adjustable, robust and well controlled tool to weaken areas of the exterior surface. The highly adjustable feature of using a laser enables laser settings to be optimized for each biomaterial and laser source in order to achieve creation of weakened areas or depressions, but not through holes. Thermal changes causes by the laser result in the creation of the weakened areas or depressions. This ensures that preferential degradation occurs while the overall biomaterial integrity is not compromised and continues to provide physical support for regenerating tissue.

In the implantable medical device, the device may be tubular.

In the implantable medical device, the device may be a nerve guidance conduit. In this way, the invention allows controlled degradation over multiple stages as regeneration through a nerve guidance conduit proceeds. Tissue growth enters the proximal and distal end of the conduit and progresses to meet mid-way, thus the new tissue cable matures from the ends towards the middle of the conduit. Staged opening of channels in the conduit from the proximal and distal end would allow for appropriately timed vascularisation of the regenerating tissues.

In the implantable medical device, the bioresorbable conduit may be comprised of collagen.

In the implantable medical device, the bioresorbable conduit may have an internal diameter from about 0.5 mm to about 20 mm.

In the implantable medical device, the bioresorbable conduit may have a length from about 1 mm to about 200 mm.

In the implantable medical device, the one or more weakened areas may not be through-holes between the exterior surface and the interior surface of the bioresorbable conduit. This ensures that preferential degradation occurs while the overall biomaterial integrity is not comprised and continues to provide physical support for regenerating tissue.

In the implantable medical device, the one or more weakened areas may extend less than half the thickness of the bioresorbable conduit from the exterior surface towards the interior surface. The one or more weakened areas may be provided in a manner which creates weakened areas or depressions, but not through holes. This ensures that preferential degradation occurs while the overall biomaterial integrity is not compromised and continues to provide physical support for regenerating tissue.

In the implantable medical device, an area of the exterior surface may have a different density and/or degree of weakening of weakened areas than another area of the exterior surface.

In the implantable medical device, the density of the weakened areas near the distal ends of the bioresorbable conduit may be higher than the density of weakened areas around the middle of the length of the bioresorbable conduit.

In the implantable medical device, the degree of weakening of weakened areas near the distal ends of the bioresorbable conduit may be higher than the degree of weakening of weakened areas around the middle of the length of the bioresorbable conduit.

According to another aspect of the present invention, there is provided a method of manufacturing an implantable medical device. The method comprising: providing a bioresorbable conduit comprising an exterior surface and an interior surface; and weakening one or more areas of the exterior surface of the bioresorbable conduit, such that the one or more weakened areas degrade preferentially to the remainder of the bioresorbable conduit.

Advantageously, this aspect of the present invention provides a rapid, robust, precise and high throughput method to manufacture a device that controls the degradation of implanted biomaterials and improves tissue repair. This method is a simpler way to manufacture a device that increases vascularization of biomaterials, while avoiding the pitfalls of other methods such as using vasogenic growth factors, conduits seeded with endothelial cells, etc., as set out above.

In the method of manufacturing an implantable medical device, weakening one or more areas on the exterior surface of the bioresorbable conduit may comprise applying chemical treatment, mechanical treatment or heat treatment to one or more areas of the exterior surface of the bioresorbable conduit, such that the one or more weakened areas are created, and form one or more weakened areas configured to degrade preferentially to the remainder of the bioresorbable conduit. In this way, the one or more weakened areas may be provided in a manner which creates weakened areas or depressions, but not through holes. This ensures that preferential degradation occurs while the overall biomaterial integrity is not compromised and continues to provide physical support for regenerating tissue.

In the method of manufacturing an implantable medical device, weakening one or more areas on the exterior surface of the bioresorbable conduit may comprise applying a laser of a first wavelength and a first intensity for a first time period to one or more areas of the exterior surface of the bioresorbable conduit, such that the one or more weakened areas are created, and form one or more weakened areas configured to degrade preferentially to the remainder of the bioresorbable conduit. In this way, the one or more weakened areas may be provided in rapid, robust, precise and high throughput manner. The laser used for the laser treatment may be a non-invasive, single-step, highly adjustable, robust and well controlled tool to weaken areas of the exterior surface.

In the method of manufacturing an implantable medical device, the method may further comprise determining, based on a material of the bioresorbable conduit, the optimal values of the first wavelength, the first intensity, and the first time period of the laser to form the one or more weakened areas. In this way, the laser settings may be optimized for each biomaterial and laser in order to achieve creation of weakened areas or depressions, but not through holes. This ensures that a device is produced in which preferential degradation occurs while the overall biomaterial integrity is not compromised and continues to provide physical support for regenerating tissue.

In the method of manufacturing an implantable medical device, the bioresorbable conduit may be comprised of Collagen I; the first wavelength may be between 157 nm and 571.699 μm; the first intensity may be between 1 mW and 1 W; and the first time period may be between 1 femtosecond and 60 seconds.

In the method of manufacturing an implantable medical device, weakening one or more areas on the exterior surface of the bioresorbable conduit may comprise one or more of: leaching one or more areas on the exterior surface of the bioresorbable conduit; reducing crosslinked sections in one or more areas on the exterior surface of the bioresorbable conduit; increasing denatured created weakened sections in one or more areas on the exterior surface of the bioresorbable conduit; and incorporating degradable polymers in the exterior surface of the bioresorbable conduit.

According to another aspect of the present invention, there is provided an implantable medical device. The device comprises a first surface and a second surface. The first surface comprises one or more weakened areas configured to degrade preferentially to the remainder of the first surface. An area of the first surface has a different density and/or degree of weakening of one or more weakened areas than another area of the first surface.

It will be understood by a skilled person that any apparatus feature described herein may be provided as a method feature, and vice versa. It will also be understood that particular combinations of the various features described and defined in any aspects described herein can be implemented and/or supplied and/or used independently.

Moreover, it will be understood that the present invention is described herein purely by way of example, and modifications of detail can be made within the scope of the invention.

### BRIEF DESCRIPTION OF DRAWINGS

One or more embodiments will now be described, purely by way of example, with reference to the accompanying figures, in which:

FIG. 1 shows a schematic diagram illustrating an implantable medical device according to an embodiment.

FIG. 2 shows a flow diagram setting out a method of manufacturing an implantable medical device according to an embodiment.

FIG. 3A shows a scanning electron microscopy image of a depression in the outer surface of a collagen conduit. FIG. 3B shows a side-view of the same depression.

FIG. 4 shows a graph showing how Raman spectroscopy was used to detect the energy shift in amide bonds of collagen before and after laser treatment.

FIG. 5A shows a graph of laser exposure times against the resulting depression diameter. FIG. 5B shows a graph of laser exposure times against the resulting depression depth.

FIG. 6 shows scanning electron microscopy and confocal microscopy images of a conduit exposed to a laser.

FIG. 7A shows a graph of ultimate tensile strength comparison between a conduit with depressions and a conduit with no depressions. FIG. 7B shows a graph of amino acids release over time between a conduit with depressions and a conduit with no depressions. FIG. 7C shows a graph of changes in depression size with degradation over time.

FIG. 8 shows a graph of a count of vessels in a nerve guidance conduit with depressions and a nerve guidance conduit without depressions.

FIG. 9 shows a graph of the percentage of degraded conduits with no depressions (left) and the percentage of degraded conduits with depressions (right), highlighting that overall integrity of the conduits was not affected by depressions.

FIG. 10 shows a toluidine blue stained cross-section of a nerve guidance conduit with no depression (left) and one depression (right) at 8 weeks after implantation in a rat model of sciatic nerve injury. After implantation the depression in conduit with depressions extended and deepened into a hole (in circle).

FIG. 11A shows a graph of the quantification of vessels inside an internal matrix in a rat model of sciatic nerve injury. FIG. 11B shows a graph of quantification of vessels in the outer conduit from toluidine blue stained sections in a rat model of sciatic nerve injury. Significantly more vessels were found in the outer shells of conduits with depressions when compared to conduits with no depressions.

FIG. 12A shows a toluidine blue stained sections depicting myelinated axons in healthy nerve, conduit without depressions, and conduit with depressions. FIG. 12B shows a graph of quantification of axons. FIG. 12C shows a graph of distribution of axonal diameters.

FIG. 13A shows a transmission electron microscopy image of the details of myelin sheath in increasing magnifications. FIG. 13B shows a graph of quantification of the g-ratio. FIG. 13C shows a linear regression curve of the g-ratio and axon diameter.

FIG. 14A shows a graph of sensory recovery as % of healthy un-operated leg at 8 weeks after implantation in a rat model of sciatic nerve injury.

FIGS. 14B and 14C show graphs of electrophysiological measurements of the example experiment.

FIG. 15A shows a graph of quantification of muscle weight. Significantly more muscle was preserved in conduits with depressions when compared to conduits with no depressions and animals without any conduit (empty defect). FIG. 15B shows the tibialis anterior muscles of operated and un-operated legs.

FIG. 16A shows toluidine blue stained mid-sections of conduits showing conduit degradation in time in a rat and rabbit in vivo model. FIG. 16B shows a graph of quantification of the percentage of degraded conduits in a rat 8-week study and a rabbit 6- and 12-week study.

FIG. 17A shows a graph of quantification of the number of myelinated axons. Significantly more myelinated axons were present in conduits with depressions when compared to conduits with no depressions. FIG. 17B shows toluidine blue stained mid-sections of conduits.

FIG. 18A shows tibialis anterior muscle in rabbit in vivo study at 12 weeks after implantation. FIG. 18B shows a graph of quantification of the muscle cross-section area. Significantly more muscle was preserved in conduits with depressions when compared to conduits with no depressions.

### DETAILED DESCRIPTION

In the following description and accompanying drawings, corresponding features may preferably be identified using corresponding reference numerals to avoid the need to describe said common features in detail for each and every embodiment.

For clarity and brevity, the terms “exterior”, “interior”, refer to relative positions as depicted in the figures. It will be appreciated that these terms do not require that any of the embodiments described herein may only be operated in a particular orientation.

Furthermore, unless explicitly specified otherwise, terms such as “located”, “positioned”, “disposed” are merely intended to express relative position of two components, and do not exclude other components from being located between said two components.

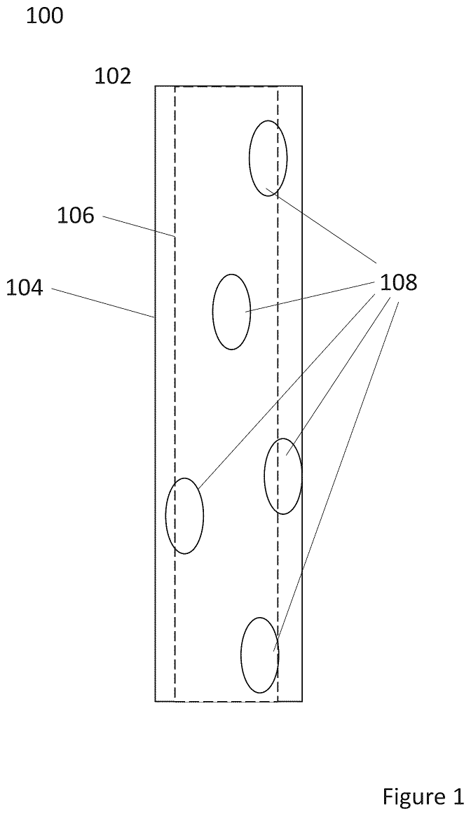

FIG. 1 shows an implantable medical device 100 according to an embodiment of the present disclosure.

The implantable medical device 100 may be comprised of biomaterials. Biomaterials are biocompatible and may be natural or synthetic materials, including natural polymers and synthetic polymers. The invention may be utilized in numerous devices composed of biomaterials that would benefit from controlled degradation. Preferably, the implantable medical device is a nerve guidance conduit which may be used for nerve regeneration. However, the skilled person will readily understand that the embodiments described can also be used with other implantable medical devices, such as biomaterials developed for repairing damaged tissue or delivering therapeutics for a myriad of applications such as musculoskeletal, cardiovascular, ocular, skin and other soft tissue, gastrointestinal, respiratory and neural indications.

The implantable medical device 100 includes a bioresorbable conduit 102 which has an exterior surface 104 and an interior surface 106. The bioresorbable conduit 102 may be comprised of biomaterials. The exterior surface 104 further includes one or more weakened areas 108. These one or more weakened areas 108 are configured to degrade preferentially to the remainder of the bioresorbable conduit 102.

The implantable medical device 100 may further comprise a material located within the limits of, but separate to, the bioresorbable conduit 102. The material may be a matrix contained within the limits of the bioresorbable conduit 102. The matrix may comprise collagen, fibronectin, laminin-1, laminin-2 and/or other macromolecules. The matrix may be bioresorbable.

The term “bioresorbable” takes its usual definition of the art, and so refers to the ability to degrade in vivo. The bioresorbable conduit may be cross-linked, and the skilled person will appreciate that the rate of absorption in vivo of the conduit as a whole may be controlled by the degree of cross-linking imparted by chemical or physical treatment. The cross-linking can be done with chromium sulfate, formaldehyde, glutaraldehyde, carbodiimide, adipyl dichloride, and the like. Factors controlling the extent of crosslinking are the type and concentration of the cross-linking agent, the pH, and the temperature of incubation. The bioresorbable conduit may be comprised of collagen, which may possess intra- or intermolecular cross-linking. The bioresorbable conduit may even consist of collagen. As will be understood by the skilled person, collagen is a protein mostly found in fibrous tissue and constitutes the major protein of various connective tissues in the body. The collagen of the bioresorbable conduit may be Type I, Type II, Type III, Type IV, or Type V collagen, and may preferably be Type I collagen. The collagen of the bioresorbable conduit may also be alkali-treated collagen. The bioresorbable conduit may comprise pores, which may for example have an average diameter of about 20 μm to about 200 μm, about 40 μm to about 180 μm, about 60 μm to about 160 μm, about 80 μm to about 140 μm, or about 100 μm to about 120 μm.

The term “conduit” takes its usual definition in the art and so refers to a three-dimensional shape with a cross section of any two-dimensional shape which allows communication between two different regions. For example, it can refer to a hollow, open ended tube or trough. In a preferred embodiment, the conduit is tubular. The conduit may have a longitudinal axis, which will be understood as referring to the axis that extends lengthways along the conduit, from one end of the shape to the other. The conduit may be open along its longitudinal axis, and may be formed by a sheet. The implantable medical device may be a nerve guidance conduit. In an embodiment in which the implantable medical device is a nerve guidance conduit, once the device is implanted in the body and positioned between two stumps of a severed nerve, the longitudinal axis of the conduit extends between the two stumps (and so corresponds with the axis of the severed nerve).

The implantable medical device may be in any three-dimensional shape, for example, generally cylindrical or tubular, or planar such as a sheet or membrane having a first surface and a second surface and having a thickness between the first surface and second surface. The descriptions herein about an exterior surface and an interior surface of a conduit are applicable to a first surface and a second surface of a medical device which is planar or in another shape. The exact dimensions and shape of the implantable medical device can be tailored depending on the circumstances of use. For example, it can be of any shape that can act as a barrier to prevent cellular migration across the material, that can be modified to accelerate focal degradation and provide portal for cellular transit through the barrier. For example, in the embodiment in which the implantable medical device is a nerve guidance conduit, it can be tailored depending on the particular nature of the nerve damage to be repaired. For example, the length of the nerve guidance conduit can be tailored depending on the length of the nerve gap to be bridged, and the diameter of the nerve guidance conduit can be tailored depending on the diameter of the damaged nerve. The resorption rate of the nerve guidance conduit as a whole can also be varied as desired. For example, the nerve guidance conduit may be tailored (by cross-linking of the materials in question) to such an extent that they are completely resorbed within about 1 to about 3 months. The degradation time and rate of implantable medical devices must be finely tuned with tissue regeneration, as for example, it has been shown that prolonged degradation of nerve guidance conduits obstructs nerve regeneration.

The total length of the implantable medical device refers to the full length extending from one terminus of the implantable medical device to the other. The total length of the implantable medical device can vary from about 1 mm to about 200 mm, from about 1 mm to about 100 mm, from about 10 mm to about 50 mm, from about 17 mm to about 38 mm, or from about 20 mm to about 38 mm.

The length of the bioresorbable conduit can be tailored depending on the circumstance of use, and can vary from about 1 mm to about 200 mm, from about 1 mm to about 100 mm, from about 10 mm to about 50 mm, from about 17 mm to about 38 mm, or from about 20 mm to about 38 mm. The inner diameter of the bioresorbable conduit can be tailored depending on the circumstance of use, and can vary from about 0.5 mm to about 20 mm, from about 0.5 mm to about 15 mm, from about 1 mm to about 15 mm, from about 1.5 mm to about 10 mm, from about 1.5 mm to about 7.0 mm, or from about 2 mm to about 7 mm. The wall thickness of the bioresorbable conduit can vary and can be tailored to balance a desired permeability with enough compressive strength to prevent collapse. The bioresorbable conduit may have walls as thin as possible while still withstanding suturing and collapse when used in vivo. For example, the bioresorbable conduit may have a wall thickness in the range of from about 0.2 mm to about 1.2 mm, e.g. about 0.1 mm to about 0.8 mm.

The one or more weakened areas 108 can extend at various degrees through the thickness of the conduit 102 from the exterior surface 104 to the interior surface 106. For example, the one or more weakened areas may be confined to the exterior surface 104 of the bioresorbable conduit 102 and therefore do not extend through the bioresorbable conduit 102 to the interior surface 106 of the bioresorbable conduit 102. As another example, the one or more weakened areas 108 do not extend through the whole thickness of the bioresorbable conduit 102 from the exterior surface 104 to the interior surface 106. When the one or more weakened areas 108 do not extend through the whole thickness of the bioresorbable conduit 102, the one or more weakened areas 108 may, for example, extend less than half the thickness of the bioresorbable conduit 102 from the exterior surface 104 towards the interior surface 106. Alternatively, the one or more weakened areas 108 may extend less than one quarter, one third, or three quarters of the thickness of the bioresorbable conduit 102 from the exterior surface 104 toward the interior surface 106. The one or more weakened areas 108 may extend nearly the entire thickness of the bioresorbable conduit 102 but they are not through-holes between the exterior surface 104 and the interior surface 106 of the bioresorbable conduit 102. By ensuring that the one or more weakened areas 108 are not through-holes, and may be, at most depressions or indentations, the process during which such depressions, indentations, or weakened areas are enlarged into hole (due to preferential degradation) provides sufficient time for host cells infiltration and restructuring of the internal matrix. This is apparent by undisturbed cell density and distribution within conduits with weakened areas which is similar to conduits without weakened areas, as shown in FIG. 9. The overall integrity of the conduit is preserved and provides a shielded environment from excess immune cells and fibroblasts and physical support for regenerating tissue.

It will be understood that the term “degrade preferentially’ refers to the ability of the weakened area of bioresorbable material to degrade before the remainder of the bioresorbable material. Furthermore, “degrade preferentially” may mean that the first stage degradation of the weakened areas of bioresorbable material starts before the second stage degradation of the remainder of the bioresorbable material, but does not necessarily mean that the first stage of degradation finishes before the second stage of degradation begins.

The diameter of each of the one or more weakened areas 108 may vary depending on the circumstance of use and the desired outcome. The diameter of each of the one or more weakened areas 108 can vary from about 1 μm to about 2000 μm, from about 1 μm to about 1000 μm, from about 50 μm to about 500 μm, from about 100 μm to about 300 μm, or from about 150 μm to about 250 μm.

The depth of each of the one or more weakened areas may vary depending on the circumstance of use and the desired outcome. The depth of each of the one or more weakened areas 108 can vary from about 1 μm to about 250 μm, from about 1 μm to about 150 μm, from about 1 μm to about 20 μm, or from about 20 μm to about 150 μm.

The one or more weakened areas 108 of the exterior surface 104 of the bioresorbable conduit 102 may be evenly distributed on the exterior surface 104 or located within one or more of concentrated areas of the exterior surface 104. An area of the exterior surface 104 may have a different density and/or degree of weakening of weakened areas 108 than another area of the exterior surface 104. Distribution of weakened areas may be focused on the distal ends or periphery of the bioresorbable conduit 102. For example, the density of weakened areas near the distal ends the bioresorbable conduit 102 is higher than the density of weakened areas around the middle of the length of the bioresorbable conduit 102. Alternatively, the degree of weakening of weakened areas near the distal ends the bioresorbable conduit 102 may be higher than the degree of weakening of weakened areas around the middle of the length of the bioresorbable conduit 102. In this way, faster initiation of the degradation may be allowed, as well as faster cell growth from the ends of severed nerves towards the middle of the bioresorbable conduit 102. Distribution of one or more weakened areas may alternatively be focused in the middle of the bioresorbable conduit 102. The one or more weakened areas 108 may not cover a large area of the exterior surface 104 of the bioresorbable conduit 102, as this may lead to too fast degradation and increase immune cell and fibroblast infiltration that would result in prolonged inflammatory phase and foreign body reaction. The spatial distribution of the one or weakened areas may be optimised to provide structure integrity while allowing the multi-stage degradation. The one or more weakened areas may be positioned along one axes of symmetry of the bioresorbable conduit 102 or two axes of symmetry of the bioresorbable conduit 102. The one or more weakened areas may be arranged in a pattern of different densities and/or weakening degrees within the bioresorbable conduit 102 to allow for phased or programmed degradation.

The one or more weakened areas 108 of the exterior surface 104 of the bioresorbable conduit 102 may not result in visible changes to the structure of the bioresorbable conduit 102. Alternatively, or additionally, the one or more weakened areas 108 of the exterior surface 104 of the bioresorbable conduit 102 may be surface depressions or indentations. The surface depressions may or may not be visible. The term “depression” takes its usual definition in the art and so refers to an area in a surface which is sunken, depressed below, or lower than the surrounding area but is not a through hole. As such, said depressions do not extend all the way between the exterior surface 104 and the interior surface 106 of the bioresorbable conduit 102. Said depressions may extend less than half the thickness of the bioresorbable conduit 102 from the exterior surface 104 towards the interior surface 106.

Alternatively, or additionally, the one or more weakened areas 108 of the exterior surface 104 of the bioresorbable conduit 102 may be indents, craters, and/or pits, but not through holes.

The one or more weakened areas 108 may be obtainable by chemically-treating, mechanically-treating or heat-treating the exterior surface 104. Localised heat mediated denaturation includes without limitation the use of laser, for example, applying a concentrated laser beam in a single location of the material. The one or more weakened areas 108 may be obtainable by laser-treating the exterior surface 104. It will be understood that when laser is applied to the exterior surface 104, the effects of the laser-treatment may not solely impact the exterior surface of the bioresorbable conduit, but may also impact the whole thickness of the bioresorbable conduit to different extents. The effects of the laser-treatment may, for example, extend over and/or into the exterior surface 104 of the bioresorbable conduit 102. Alternatively, or additionally, effects may, for example, extend between the exterior surface 104 and the interior surface 106 of the bioresorbable conduit 102. For example, the effects may extend less than half the thickness of the bioresorbable conduit 102 from the exterior surface towards the interior surface. Alternatively, the effects may extend less than one quarter, one third, or three quarters of the thickness of the bioresorbable conduit 102 from the exterior surface 104 toward the interior surface 106. Chemically-treating to obtain the one or more weakened areas may comprise using a material in specific areas of the conduit which dissolves preferentially in vivo or may be more susceptible to enzymatic degradation. For example, using gelatin in these specific areas, while the rest of the conduit is made from collagen, or using solvent to leach out a pre-determined percentage of polymer. Mechanically-treating to obtain the one or more weakened areas may comprise tethering or moulding. Moulding may comprise creating indentations using different moulds. Heat-treating to obtain the one or more weakened areas may comprise applying heat in specific areas.

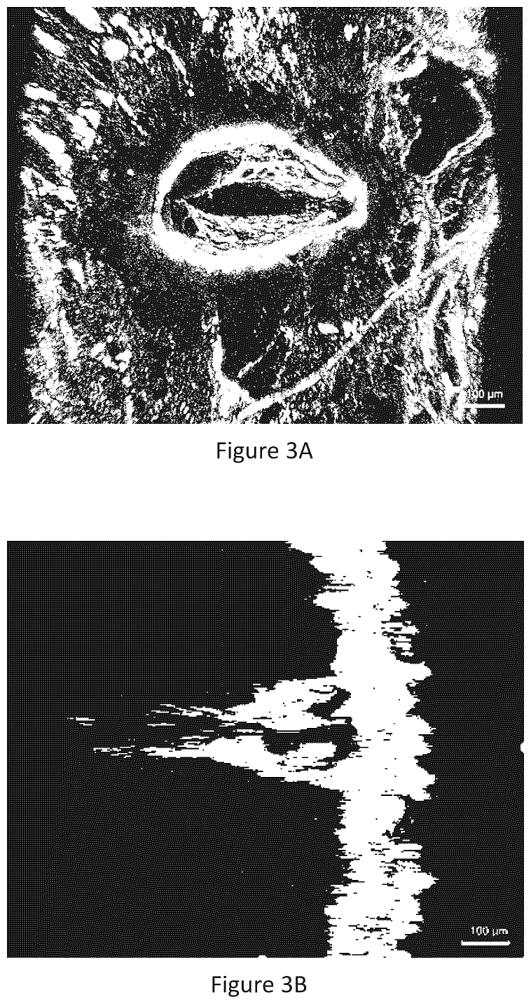

The laser used for the laser treatment may be a non-invasive, single-step, highly adjustable, robust and well controlled tool to weaken areas of the bioresorbable conduit 102. Thermal changes caused by the laser result in the creation of the weakened areas or depressions. The laser may create consistent depressions, as shown in FIGS. 3A and 3B. FIG. 3A shows a scanning electron microscopy image of an example depression in the outer surface of a collagen conduit. FIG. 3B shows a side-view of the same depression, demonstrating that it is not a through hole. In this example, a Verdi V12 laser was used at 532 nm and 800 mW to create localised degradation following 30 seconds of exposure per area of the Collagen-I based conduit.

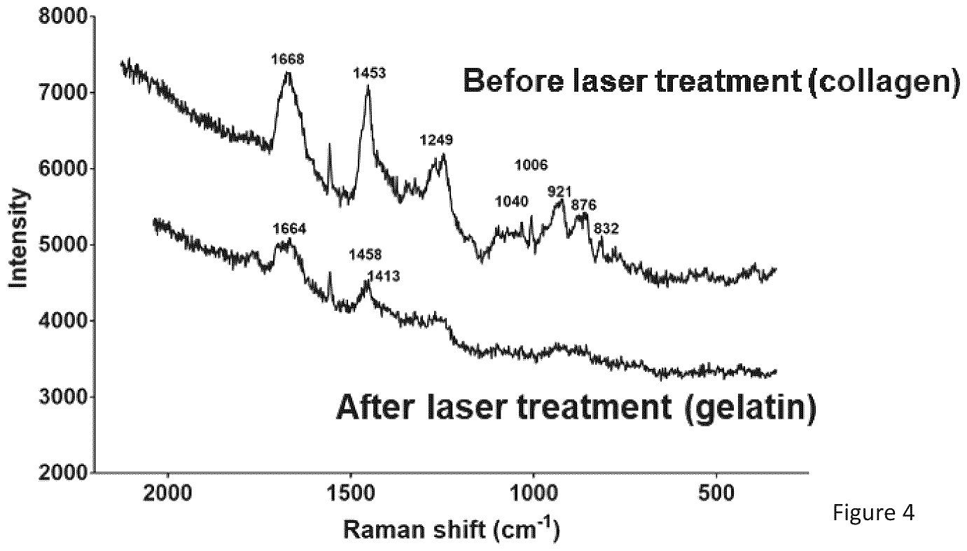

The mechanism of action of laser interaction with biomaterial is exerted by photons as a photochemical or photothermal mechanism which results in material ablation. In the photochemical mechanism, the photon energy of the light is used to break the chemical bonds of the polymer directly whereas in the photothermal mechanism the material is ablated by melting and vaporizing. For photochemical ablation to occur, energy of the photons at that wavelength should overcome the intermolecular bond energies of the polymer. The relation between the photon energy of light and laser wavelength is:

$$E = \frac{1.245}{λ} ;$$

where E is the photon energy and λ is the laser wavelength. Therefore, depending on the chemical structure of the biomaterial, suitable laser parameters are selected. To effect localized degradation of the collagen, the laser output parameters can be experimentally optimized. For the example in FIGS. 3A and 3B, the laser energy must overcome C—N amide bond with energy of 3.04 eV (˜305 KJ/mol). Following treatment using a 532 nm laser and analysis using Raman spectroscopy, the shift in amide groups of conduit before and after laser treatment is shown in FIG. 4 that is indicative of collagen degradation. FIG. 4 shows that peaks corresponding to amide I (1668 cm-1), amide II (1453 cm-1) and amide III groups (1249 cm-1) are noticeably diminished after laser treatment. Laser settings may be optimized for each biomaterial and laser in order to achieve creation of weakened areas or depressions, but not through holes. This ensures that preferential degradation occurs while the overall biomaterial integrity is not compromised and continues to provide physical support for regenerating tissue.

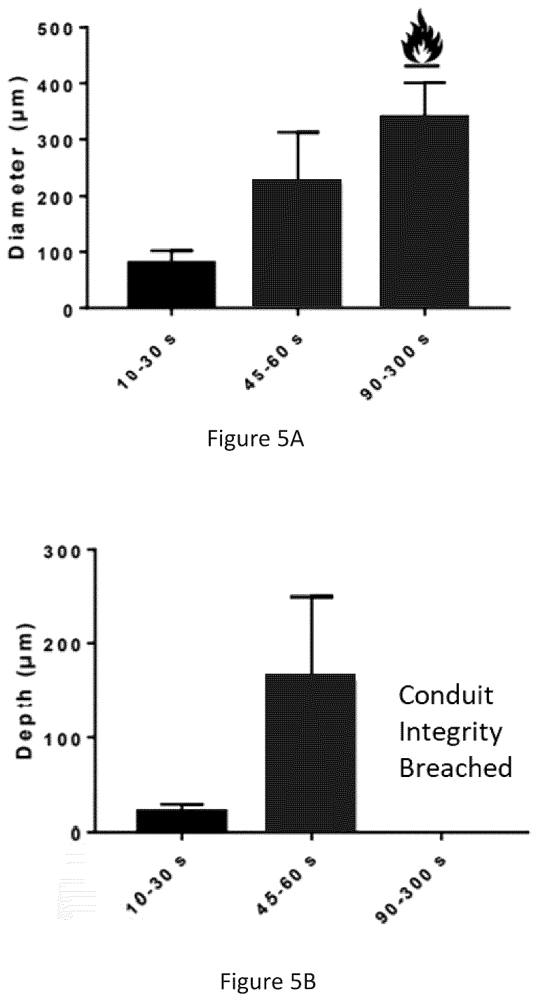

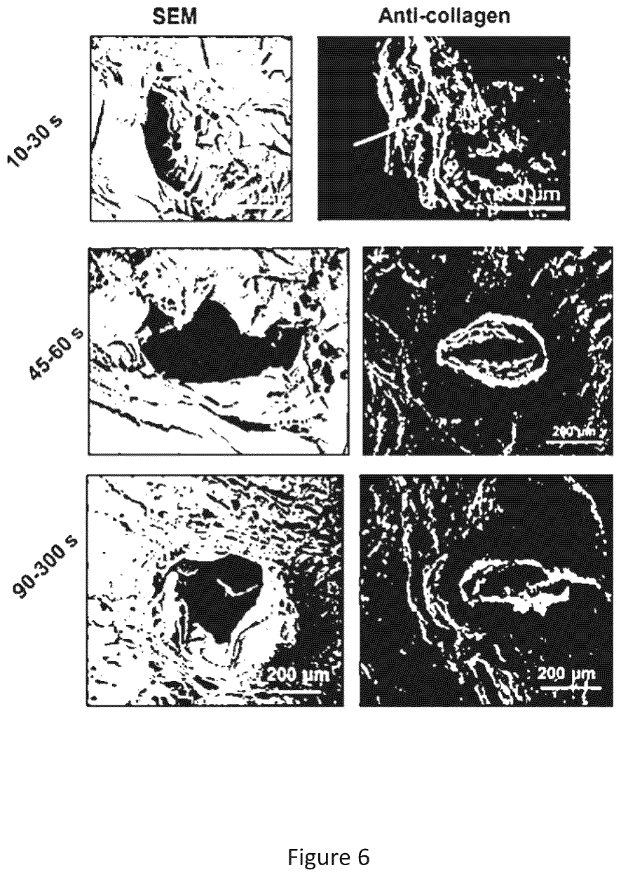

FIGS. 5A, 5B and 6 show example experiments for the optimization of laser exposure times for the creation of the weakened areas. The exposure conditions are experimentally optimized to balance localized degradation and depression formation while maintaining conduit functionality. In the above describe example, 30 seconds exposure was chosen as it caused the least disruption. This is determined based on the relatively small depressions created as well as their low depth of penetration into the collagen conduit. FIG. 5A shows in an example that extended exposure times result in larger diameter depression formation and heat damage at highest exposure times (indicated by the flammable sign). FIG. 5B shows in an example that extended laser exposure result in deeper depression formation and total loss of conduit integrity. FIG. 6 shows scanning electron microscopy on the left and confocal microscopy on the right, both examples of which reveal that only the lowest exposure time resulted in localized degradation of collagen with minimal invasive damage to the conduit structure.

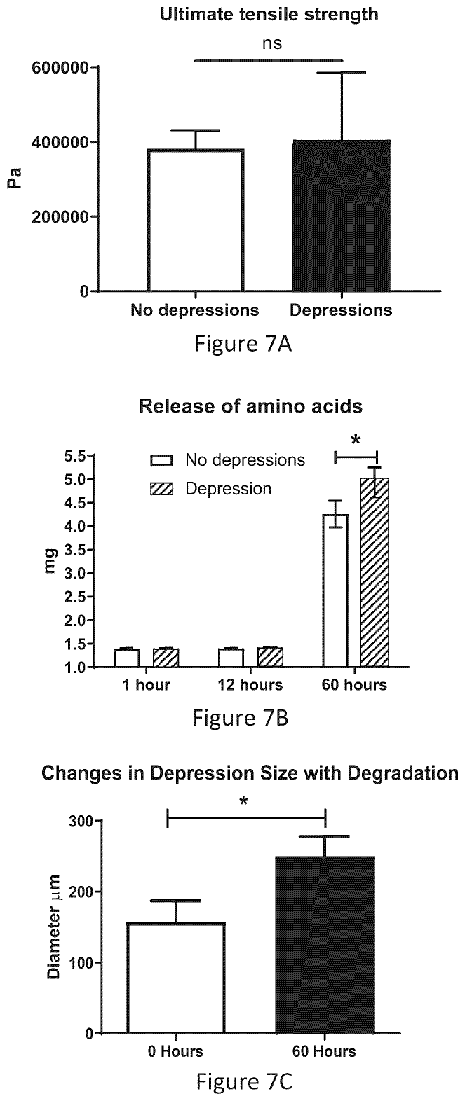

Further to these example optimization experiments, mechanical testing may be used to demonstrate that the depressions made do not compromise the integrity of the conduits. FIG. 7A shows in an example that conduits with depressions made with a 30 second exposure or without depressions at all has similar ultimate tensile strength. This also indicates that conduits with depressions will fulfil their initial role to shield the regenerating tissues from excess infiltration of immune cells and fibroblasts. On the other hand, FIGS. 7B and 7C show in an example that depressions on the exterior surface of the conduit enlarge in diameter and enable preferential degradation of these specific weakened areas over time. This is shown in these figures by measuring the depression diameter and the release of amino acids in a degradation assay. Preferentially early degraded areas then allow vascular cell infiltration and vessel formation.

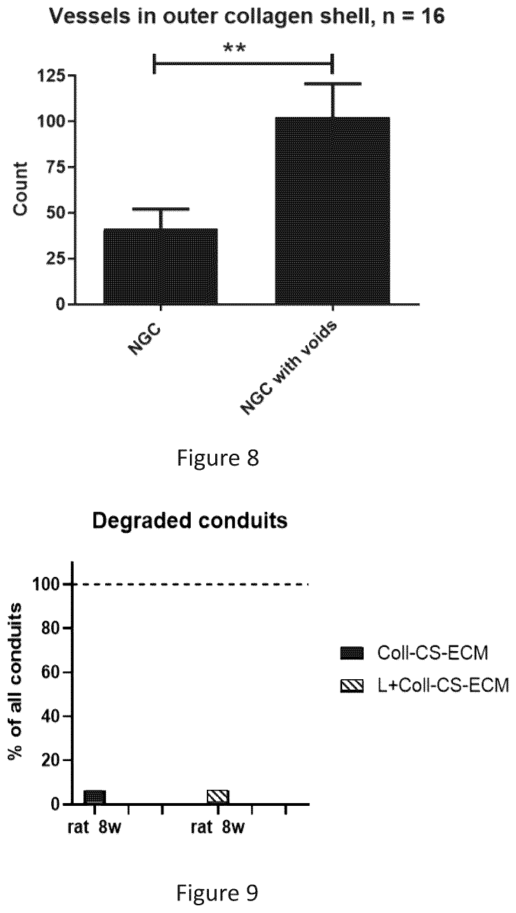

The effect of the depressions to increase vascularization was confirmed in vivo. The above described example conduits were implanted into a rat model of a peripheral nerve defect and significantly increased vascularization was observed on the exterior surface of the conduits with depressions, as demonstrated by the example vessel count in FIG. 8.

Over time, the weakened areas degrade at a faster rate than the surrounding untreated material. This allows for the creation of a concentration gradient. This will allow for the passages or substances, for example, ions, small molecules, proteins, fluids, etc. at a specific time and rate. Therefore, providing said weakened areas enables not only to control the time and rate of degradation, but also the time and rate of vascular cell infiltration and vascularisation.

Timely and appropriate degradation and vascularisation improves the overall outcome of implanted natural biomaterial. Namely increases chances for successful engrafting, minimizes host versus graft reaction, enhances nerve and other tissue regeneration.

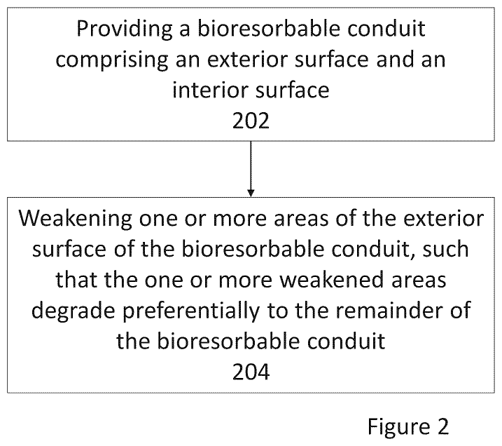

FIG. 2 sets out a method of manufacturing an implantable medical device. The method of FIG. 2 can be performed to produce the device implantable medical device of FIG. 1.

In step 202, a bioresorbable conduit 102 is provided. The provided bioresorbable conduit 102 has an exterior surface 104 and an interior surface 106.

In step 204, one or more areas of the exterior surface 104 of the bioresorbable conduit 102 are weakened, such that the one or more weakened areas 108 degrade preferentially to the remainder of the bioresorbable conduit 102.

Optionally, weakening one or more areas on the exterior surface 104 of the bioresorbable conduit 102 may further comprise applying chemical treatment, mechanical treatment or heat treatment to one or more areas of the exterior surface 104 of the bioresorbable conduit 102, such that the one or more weakened areas are created, and form the one or more weakened areas 108 configured to degrade preferentially to the remainder of the bioresorbable conduit 102. When heat treatment is applied to the one or more areas of the exterior surface 104 of the bioresorbable conduit 102, the creation of the one or more weakened areas may be as a result of denaturing or partial denaturing of the one or more areas.

Optionally, weakening one or more areas on the exterior surface 104 of the bioresorbable conduit 102 may further comprise applying a laser of a first wavelength and a first intensity for a first time period to one or more areas of the bioresorbable conduit 102, such that the one or more weakened areas are created and form the one or more weakened areas 108 configured to degrade preferentially to the remainder of the bioresorbable conduit 102.

Optionally, the optimal values of the first wavelength, the first intensity, and the first time period of the laser applied to form the one or more weakened areas 108 are determined, based on a material of the bioresorbable conduit 102.

The bioresorbable conduit 102 may be comprised of Collagen I. In this embodiment, the lower limit of the first wavelength of the laser may be at least 157 nm, at least 193 nm, at least 300 nm, or at least 500 nm and the upper limit of the first wavelength of the laser may be no more than 193 nm, no more than 300 nm, no more than 500 nm, no more than 1092 nm, no more than 2000 nm, or no more than 571.699 μm. The first wavelength of the laser can vary from about 157 nm to about 571.699 μm, from about 193 nm to about 1092 nm, or from about 157 nm to about 1092 nm. The lower limit of the first intensity of the laser may be at least 1 mW, at least 10 mW, or at least 100 mW, and the upper limit of the first intensity of the laser may be no more than 1 W, no more than 10 mW, or no more than 100 mW. The first intensity of the laser can vary from about 1 mW and 1 W, 1 mW and 100 mW, 100 mW and 1 W, or 10 mW and 100 mW. The lower limit of the first time period may be at least 1 femtosecond, at least 100 femtoseconds, at least 1 second, or at least 10 seconds, and the upper limit of the first time period may be no more than 10 femtoseconds, no more than 100 femtoseconds, no more than 1 second, no more than 10 seconds or no more than 60 seconds. The first time period can vary from about 1 femtosecond and 60 seconds, 100 femtoseconds and 1 second, 1 femtosecond and 100 femtoseconds, or 1 second and 60 seconds.

Optionally, in step 210, weakening one or more areas on the exterior surface 104 of the bioresorbable conduit 102 may further comprise one or more of leaching one or more areas on the exterior surface 104 of the bioresorbable conduit 102, reducing crosslinked sections in one or more areas on the exterior surface 104 of the bioresorbable conduit 102, increasing weakened sections in one or more areas on the exterior surface 104 of the bioresorbable conduit 102, and incorporating degradable polymers in the exterior surface 104 of the bioresorbable conduit 102.

While the foregoing is directed to exemplary embodiments of the present invention, it will be understood that the present invention is described herein purely by way of example, and modifications of detail can be made within the scope of the invention. Furthermore, one skilled in the art will understand that the present invention may not be limited by the embodiments disclosed herein, or to any details shown in the accompanying figures that are not described in detail herein or defined in the claims. Indeed, such superfluous features may be removed from the figures without prejudice to the present invention.

Moreover, other and further embodiments of the invention will be apparent to those skilled in the art from consideration of the specification and may be devised without departing from the basic scope thereof, which is determined by the claims that follow.

### EXAMPLES

### Example 1

To further demonstrate the beneficial effects on tissue regeneration through two-stage degradation, the laser-treated biomaterial was tested in vivo, in two animal models.

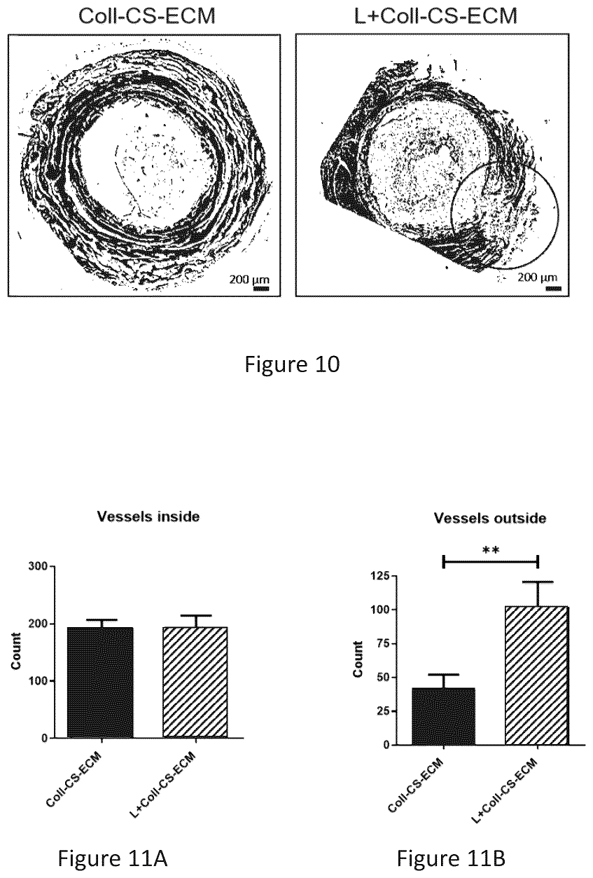

In a rat model of critical-sized sciatic nerve defect, laser-treated nerve guidance conduits (NGCs) were implanted and compared to non laser-treated NGCs. When looking at the mid-sections at about >7.5 mm, both parts of the conduits could be observed-collagen outer shell and collagen-chondroitin sulphate internal matrix. In other words, both the bioresorbable conduit and the matrix contained within the bioresorbable conduit could be observed. Although, sample cross-sections were cut at random and only some laser-treated cross-sections were made at the exact plane as the laser-treated area, it was found that in at least 31% of laser-treated samples, the laser targeted area extended and deepened to form a hole, as shown in FIG. 10. The process during which depressions/indentations enlarged into holes provided sufficient time for the host cells infiltration and restructuring of the internal matrix. This is apparent by undisturbed cell density and distribution within the laser-treated conduits which is similar to non laser-treated conduit, as shown in FIG. 9. The overall integrity of the conduit was preserved and provided shielded environment from excess immune cells and fibroblasts.

This also suggests that the conduit underwent the first stage of degradation in which the laser targeted areas underwent preferential degradation to provide space for vascular cell infiltration and enable nutrient exchange.

FIG. 10 shows toluidine blue cross-sections show intact outer shell of a non laser-treated conduit and a depression/indentation that extended into a hole in a laser-treated conduit (in circle). FIG. 9 shows the overall integrity of the conduits remained intact over the course of the study, only 6.25% of conduits completely degraded.

The first stage degradation then led to increased vascularisation-particularly in the outer part of the conduit. In the example experiments, it was found that laser-treated conduits had significantly more vessels in the outer part then non laser-treated conduits, as shown in FIGS. 11A and 11B. These vessels were found to be mature based on the positivity of CD31 staining. FIG. 11A shows quantification of vessels in the outer conduit from toluidine blue stained sections showing significant increase in vessels in laser-treated conduits. FIG. 11B shows quantification of vessels inside internal matrix shows the structure is maintained across the groups. The double asterisk, “**”, shown on FIG. 11B denotes p<0.01.

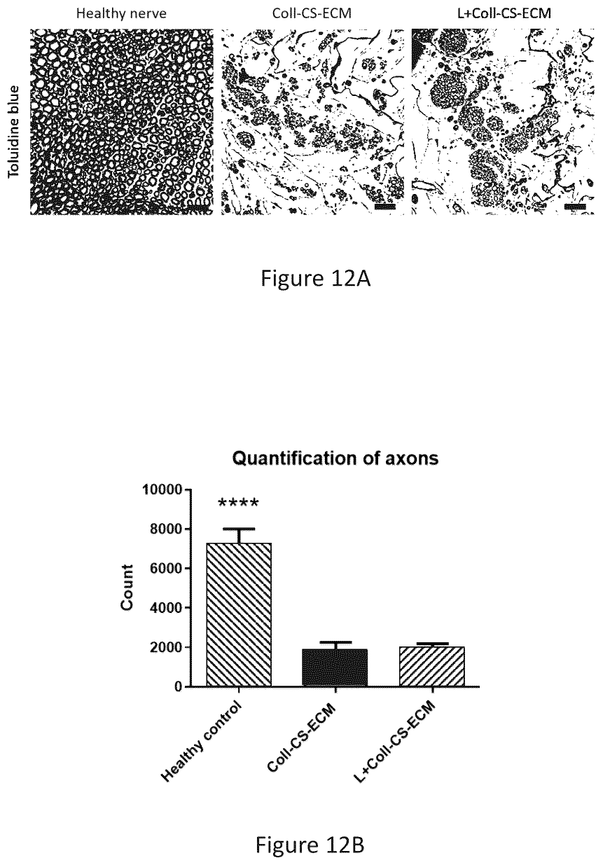

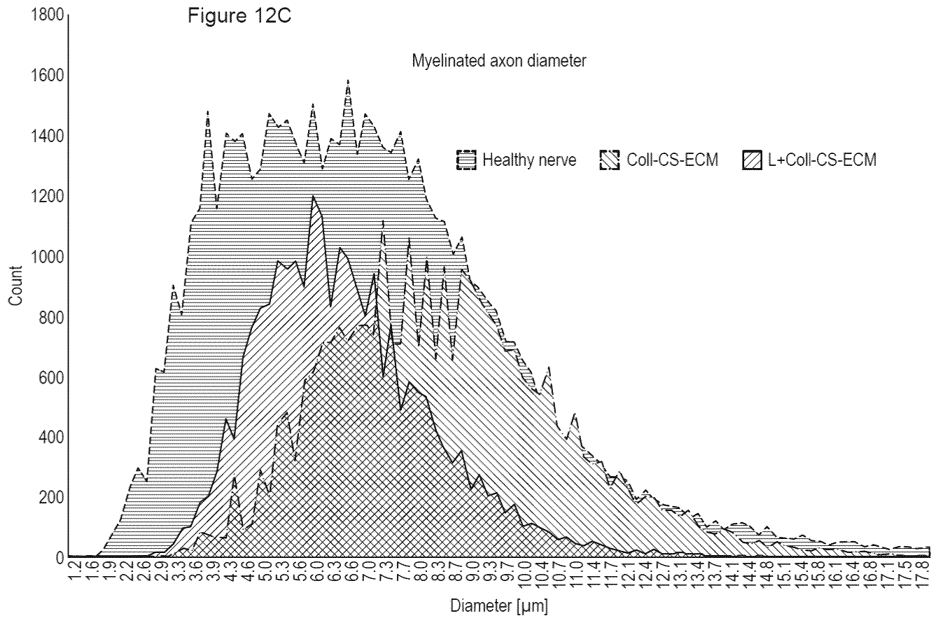

Successful nerve repair requires nerve axons to cross the gap and connect with precision to their distant targets. In the example experiments, numerous myelinated axons that grew into the conduits and extended past 7.5 mm from the proximal stump were found. Newly regenerated axons appeared in bundles and had similar morphology as in healthy un-operated nerve. On quantification of myelinated axons from toluidine blue stained sections, the number was maintained between the two tested groups, as shown in FIGS. 12A to 12C.

However, when looking at the axon calibre distribution, a difference between the two groups was observed—regenerated axons in laser-treated conduits were reminiscent in axon calibre to healthy un-operated nerves, while conduits without treatment seem to support predominantly axons with a larger calibre. This suggests that laser-treated conduits promote regeneration of whole spectrum of axons which in turn might lead to better recovery when compared to selected population of axons, as shown in FIGS. 12A to 12C.

FIG. 12A shows toluidine blue stained sections depicting myelinated axons in healthy nerve (left), non-laser treated conduit (middle), and laser-treated conduit (right). Newly regenerated axons were found to form bundles. FIG. 12B shows quantification of axons shows that the number is maintained across the tested groups (obviously, the healthy un-operated nerve contains significantly more axons). Values are shown as mean±standard error mean. FIG. 12C shows distribution of axonal diameters revealed that regenerated axons in laser-treated conduits are more native nerve alike when compared to non-laser treated conduits.

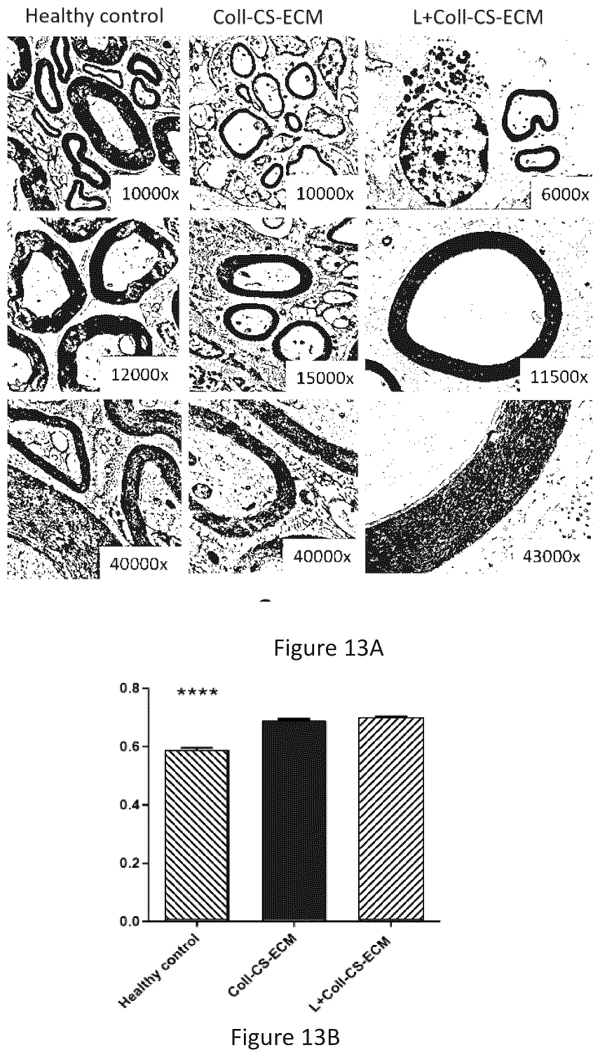

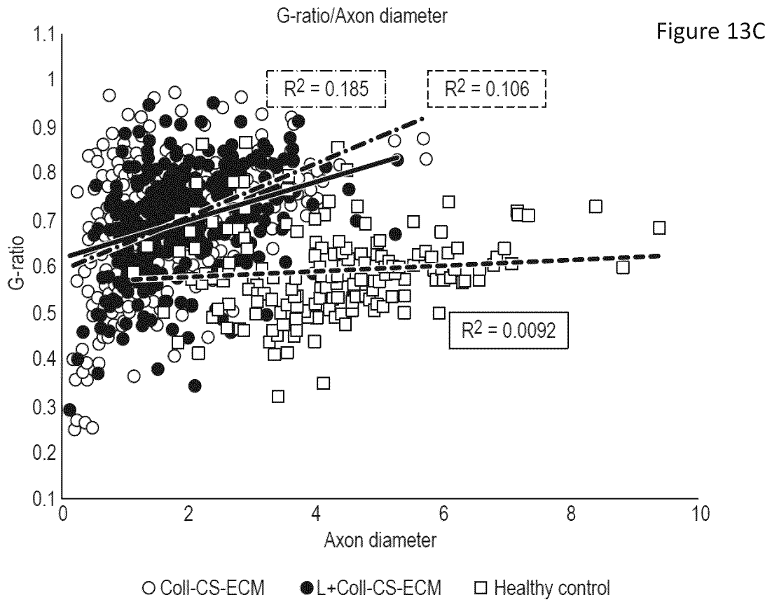

Similarly, when looking at the g-ratio quotient, which describes the relationship between axon calibre and myelinated fiber calibre for myelinated axons, it was shown that laser-treated conduits display a linear regression curve that is more similar to healthy, un-operated nerve than non laser-treated conduits. This further confirms that laser-treated conduits enable adequate myelination of newly grown axons, as shown in FIGS. 13A to 13C.

FIG. 13A shows transmission electron microscopy shows details of myelin sheath in increasing magnifications. FIG. 13B shows quantification of the G-ratio shows the number is maintained across the treatment groups. The healthy group axons have significantly lower g-ratio. Each group contains values of thousands of axons. Values are shown as mean±standard error mean. FIG. 13C shows linear regression curve of the g-ratio and axon diameter. Axons in laser-treated conduits have regression curve similar to healthy un-operated nerves.

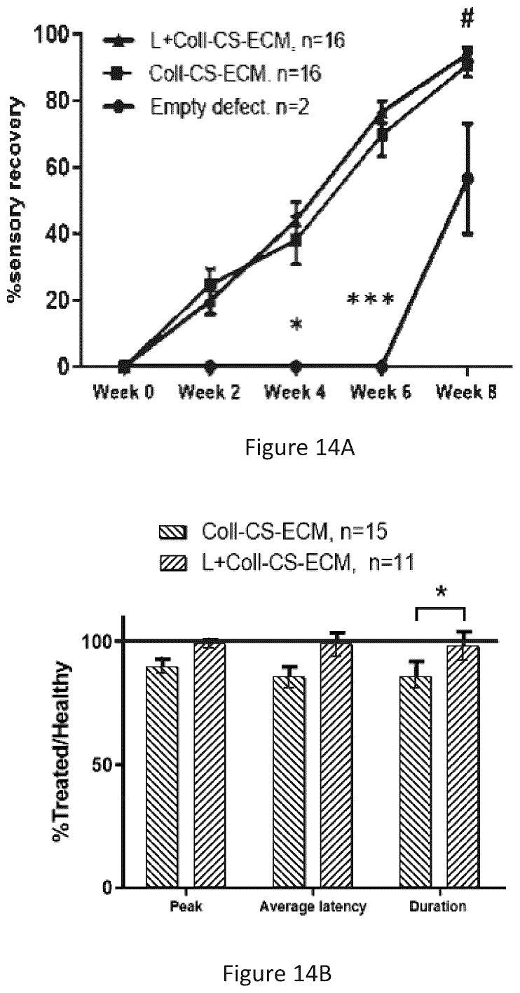

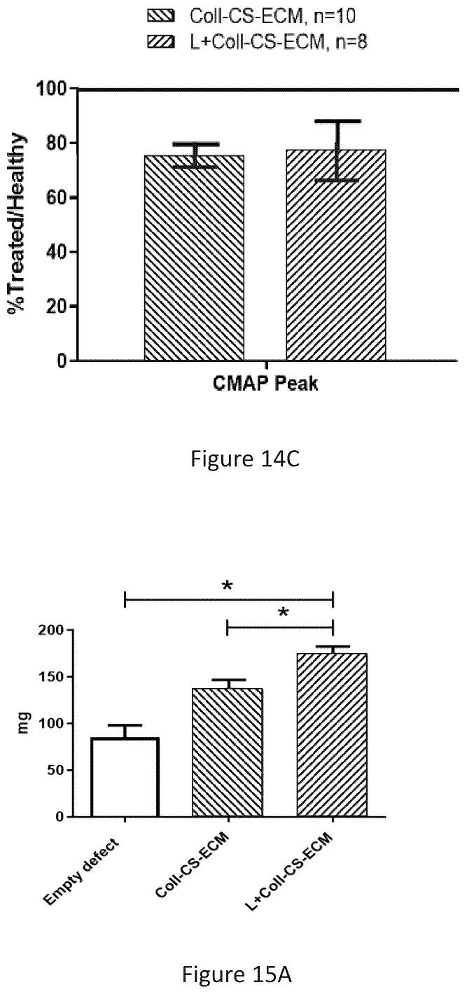

Electrophysiological measurement further confirmed functional repair. Sensation and retrieval of feet as reaction to electrical stimulus reached 94% of the healthy, un-operated leg by the 8 weeks after the surgery. The rate of sensory recovery, compound nerve action potential (CNAP) peak and CNAP average latency and compound muscle action potential (CMAP) peak were similar in both, laser-treated and non laser-treated group. Laser-treated conduits significantly improved the duration of the CNAP when compared to non-laser treated conduit and the duration was almost same as in healthy un-operated leg, as shown in FIGS. 14A to 14C.

FIG. 14A shows sensory recovery as % of healthy un-operated leg. Both treatments significantly differ at weeks 4 and 6 from an empty defect. Laser-treated group has significantly improved sensory recovery also at 8 weeks after the surgery.

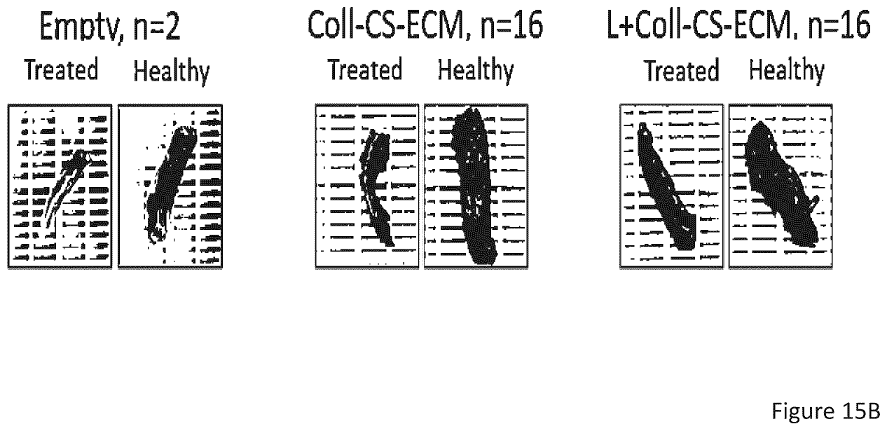

Loss of innervation in muscles causes their rapid atrophy—this was evident when comparing the weights of the tibialis anterior muscle in the presently described example experiments. In all animals the muscle on the operated leg was smaller than the one on the healthy un-operated leg. Quantification of the muscle mass showed that the laser-treated conduits helped to re-innervate and regain muscle weight significantly more when compared to non laser-treated group, as shown in FIGS. 15A and 15B.

FIG. 15A shows quantification of muscle weight and demonstrates that laser treated group had muscle mass significantly recovered when compared not only to an empty defect but also non laser-treated group. The asterisk, “*”, shown on FIG. 15A denotes p<0.05. Values are shown as mean±standard error mean. FIG. 15B shows the tibialis anterior (TA) muscles of operated and un-operated legs. Specifically, Left-TA of an animal with an empty defect (n=2), middle-TA of an animal implanted with non laser-treated conduits (n=16), right-TA of an animal implanted with laser-treated conduits (n=16).

Collectively the data from the example rat in vivo study show that when laser-treated conduits undergo the first stage of degradation, they significantly improve the vascularisation of the conduits, the myelination of axons is reminiscent of healthy nerves and their function approaches the function of a healthy nerve. Moreover, laser-treated conduits significantly preserve more muscle mass than non laser-treated conduits.

### Example 2

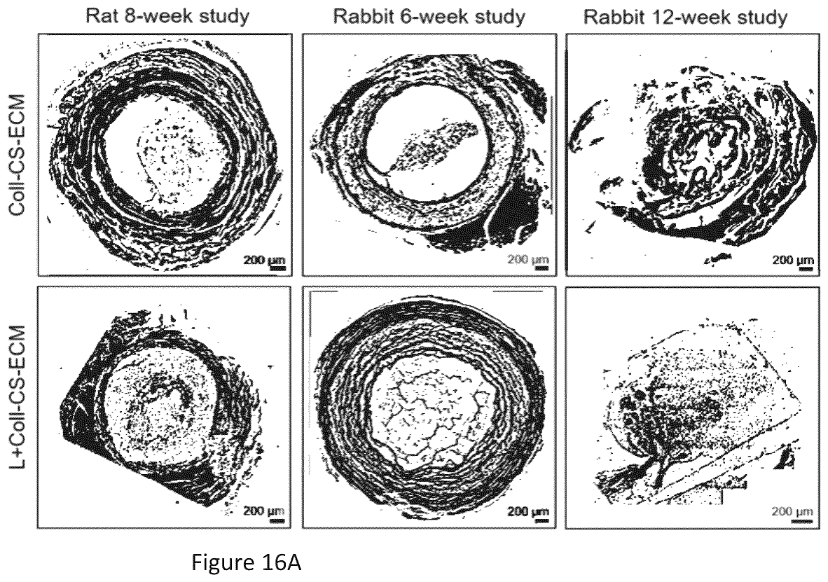

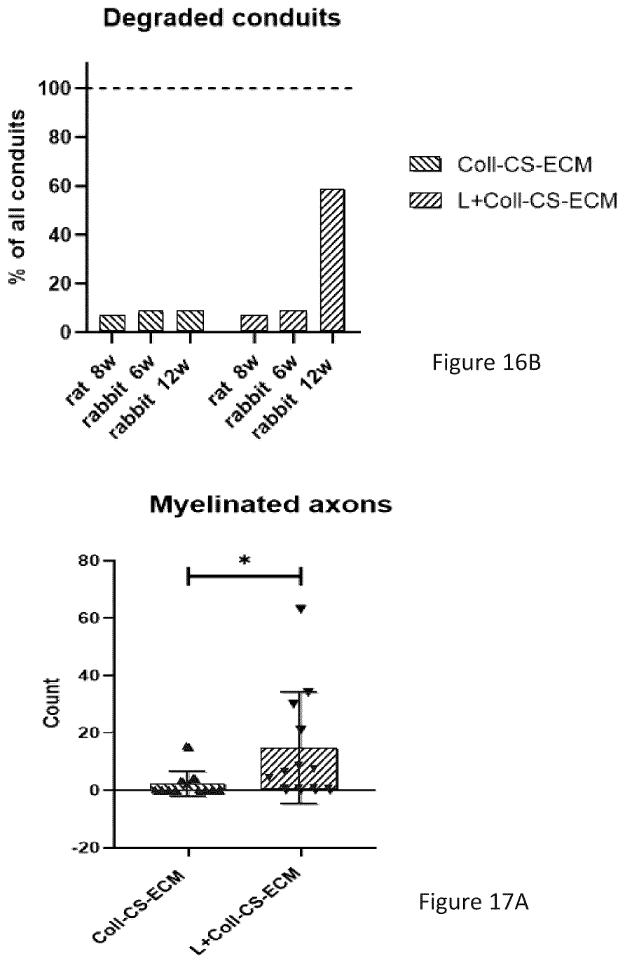

As another example experiment, laser and non laser-treated conduits were implanted in a challenging 30 mm long nerve gap in a rabbit in vivo model. Conduits were implanted for 6 and 12 weeks. Examination of the gross morphology of the conduits provided further insights about timing of the first and second stage of degradation, as shown in FIGS. 16A and 16B. The first stage of degradation that was manifested at 8 weeks after the surgeries in the rat study was also present at about 6 weeks in the rabbit study. Rabbits are metabolically highly active and therefore, the fact that the first stage of degradation took place earlier than in rats is plausible. By 12 weeks after the surgery, total degradation of outer conduit shell in about 60% of the conduits was observed. This is indicative of the second stage of degradation—the bulk degradation. Bulk degradation of conduits was matched with complete host tissue replacement, as shown in FIGS. 16 and 16B.

FIG. 16A shows conduit degradation in time in a rat and rabbit in vivo model with toluidine blue stained mid-sections of the conduits. FIG. 16B shows a quantification of the percentage of degraded conduits.

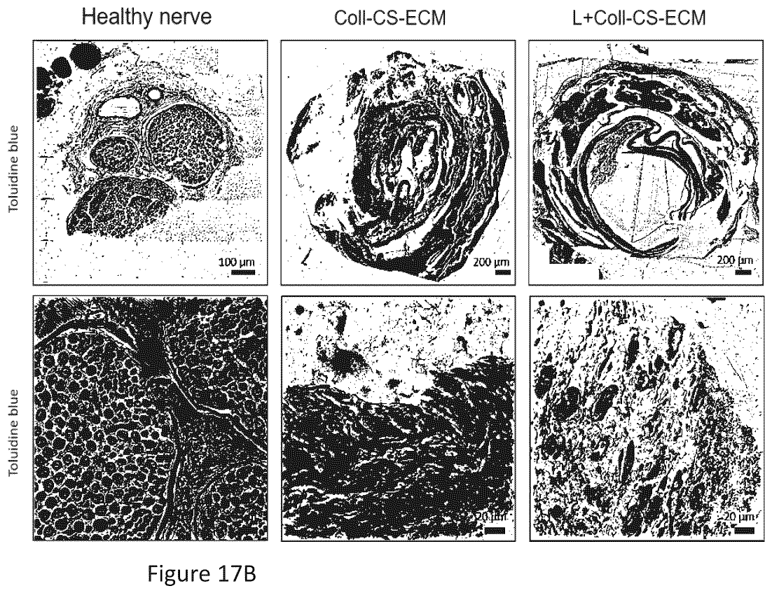

When examining toluidine blue stained mid-sections in detail, significantly more myelinated axons in the laser-treated conduits were found when compared to non laser-treated conduits, as shown in FIGS. 17A and 17B. This might suggest that the bulk degradation observed in majority of laser-treated conduits promoted increased nerve ingrowth.

Electrophysiological measurements in rabbits also revealed functional nerve repair and conduction of neural activity was recorded in distal parts of the nerve and even muscle.

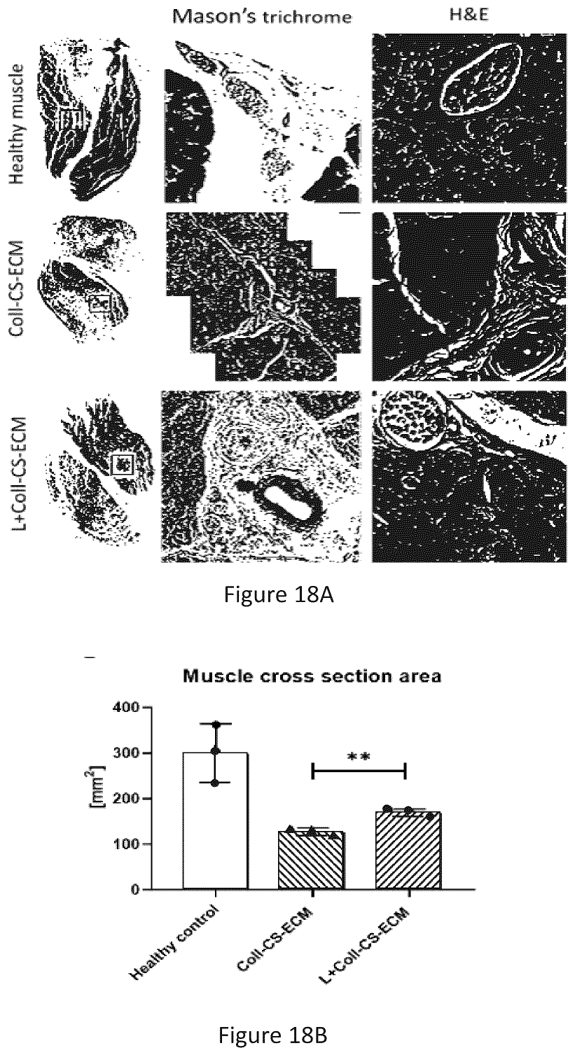

Finally, when quantifying the muscle mass area on the cross-section, the laser-treated group were found to have significantly more preserved muscle mass than non laser-treated group, as shown in FIGS. 18A and 18B. This is in line with the observations from a rat study where the laser-treated groups also preserved significantly more muscle mass.

FIG. 18A shows tibialis anterior muscle in rabbit in vivo study. Left column-cross-section of the muscle that bifurcates in two parts. Middle column—detail of Mason's trichrome staining. Right column—detail of haematoxylin and eosin staining. Empty epineuria could be observed in both histological preparations. FIG. 18B shows Quantification of the muscle cross-section area. The double asterisk, “**”, shown on FIG. 18A denotes p<0.01. Values are shown as mean±standard error mean.

Data from rabbit study example are indicative that at 12 weeks after the surgery the second stage of degradation took place. This was evident by complete conduit degradation that was matched with host tissue replacement and improved outcomes of nerve regeneration. In particular, an increased number of myelinated axons, nerve functional repair and increased preservation of muscle mass were observed. Results from the rabbit in vivo study example corroborated previous results from the rat in vivo study example.

Altogether, the results from two in vivo study examples firstly show that laser provided a method for controlled degradation of implanted biomaterials. Secondly, they show that the degradation of laser-treated conduits is carried out in multiple stages. And finally, they show that two-stage degradation is beneficial for tissue regeneration.

1. An implantable medical device comprising:
a bioresorbable conduit comprising:
an exterior surface comprising one or more weakened areas configured to degrade preferentially to a remainder of the bioresorbable conduit; and
an interior surface.

2. The implantable medical device of claim 1, wherein the one or more weakened areas comprise surface depressions.

3. The implantable medical device of claim 1, wherein the one or more weakened areas each have a diameter of 50-2000 μm.

4. The implantable medical device of claim 1, wherein the one or more weakened areas are obtainable by chemically-treating, mechanically-treating or heat-treating the exterior surface.

5. The implantable medical device of claim 1, wherein the one or more weakened areas are obtainable by laser-treating the exterior surface.

6. The implantable medical device of claim 1, wherein the device is tubular.

7. The implantable medical device of claim 1, wherein the device is a nerve guidance conduit.

8. The implantable medical device of claim 1, wherein the bioresorbable conduit comprises collagen.

9. The implantable medical device of claim 1, wherein the bioresorbable conduit has an internal diameter from about 0.5 mm to about 20 mm.

10. The implantable medical device of claim 1, wherein the bioresorbable conduit has a length from about 1 mm to about 200 mm.

11. The implantable medical device of claim 1, wherein the one or more weakened areas extend less than half of a thickness of the bioresorbable conduit from the exterior surface towards the interior surface.

12. The implantable medical device of claim 1, wherein an area of the exterior surface has a different density and/or degree of weakening of weakened areas than another area of the exterior surface.

13. The implantable medical device of claim 1, wherein:
a density of weakened areas near distal ends of the bioresorbable conduit is higher than a density of weakened areas around a middle of a length of the bioresorbable conduit; and/or
a degree of weakening of weakened areas near the distal ends of the bioresorbable conduit is higher than a degree of weakening of weakened areas around the middle of the length of the bioresorbable conduit.

14. A method of manufacturing an implantable medical device, the method comprising:
providing a bioresorbable conduit comprising an exterior surface and an interior surface; and
weakening one or more areas of the exterior surface of the bioresorbable conduit, such that the one or more weakened areas of the exterior surface degrade preferentially to a remainder of the bioresorbable conduit.

15. The method of claim 14, wherein weakening one or more areas on the exterior surface of the bioresorbable conduit comprises:
applying chemical treatment, mechanical treatment or heat treatment to one or more areas of the exterior surface of the bioresorbable conduit, such that the one or more weakened areas are created, and form the one or more weakened areas configured to degrade preferentially to the remainder of the bioresorbable conduit.

16. The method of claim 14, wherein weakening one or more areas on the exterior surface of the bioresorbable conduit comprises:
applying a laser of a first wavelength and a first intensity for a first time period to one or more areas of the exterior surface of the bioresorbable conduit, such that the one or more weakened areas are created, and form the one or more weakened areas configured to degrade preferentially to the remainder of the bioresorbable conduit.

17. The method of claim 16, the method further comprising:
determining, based on a material of the bioresorbable conduit, optimal values of the first wavelength, the first intensity, and the first time period of the laser applied to form the one or more weakened areas.

18. The method of claim 16, wherein when the bioresorbable conduit comprises Collagen I:
the first wavelength is between 157 nm and 571.699 μm;
the first intensity is between 1 mW and 1 W; and
the first time period is between 1 femtosecond and 60 seconds.

19. The method of claim 14, wherein weakening one or more areas on the exterior surface of the bioresorbable conduit comprises one or more of:
leaching one or more areas on the exterior surface of the bioresorbable conduit;
reducing crosslinked sections in one or more areas on the exterior surface of the bioresorbable conduit;
increasing created weakened sections in one or more areas on the exterior surface of the bioresorbable conduit; and
incorporating degradable polymers in the exterior surface of the bioresorbable conduit.

20. An implantable medical device comprising:
a first surface comprising one or more weakened areas configured to degrade preferentially to a remainder of the first surface; and
a second surface,
wherein an area of the first surface has a different density and/or degree of weakening of one or more weakened areas than another area of the first surface.
